(19) United States
(12) Patent Application Publication
(10) Pub. No.: US 2016/0106440 A1
(43) Pub. Date: Apr. 21, 2016

(54) SURGICAL RONGEUR WITH DISPOSABLE AND REPLACEABLE CUTTING BLADE

(71) Applicant: Mark Fischer Harper, Sarasota, FL (US)

(72) Inventor: Mark Fischer Harper, Sarasota, FL (US)

(21) Appl. No.: 14/516,741

(22) Filed: Oct. 17, 2014

(51) Int. Cl. A61B 17/16

(52) U.S. Cl. CPC A61B 17/1606

(57) ABSTRACT

A surgical cutting device is described having a main body having an integral rear handle and a forward handle connected thereto. A sliding member sits atop a rail integral with the main body such that the sliding member is also moveably attached to the forward handle. The sliding member has a forward cutting device that is easily separable, reusable, disposable and replaceable using a simple mechanical engagement of perforations in the cutting device and protrusions in the sliding member. A strike end plate acts in conjunction with the sliding cutting device to grasp and cut bio matter from a patient during surgical procedures.

### FIELD OF THE INVENTION

The present invention is generally directed to surgical devices. More particularly, the present invention is related to a type of surgical device known as a rongeur; this device is used to remove tissues and bone matter during surgery from a region of the human body.

### BACKGROUND OF THE INVENTION

A rongeur is a heavy duty device that is utilized during surgery to remove tissues and bones as needed by medical personnel. It is typically used in orthopedic or neurosurgery to gouge out bones and tissue thereby exposing structures for further surgical treatment. Other uses for rongeurs include in oral maxillofacial surgery and hand surgery to cut traumatic amputated bone. One type of rongeur is known as a Kerrison type that is used during spinal surgery to remove bone, cartilage and soft tissue from the spinal posterior in order to gain access into the spinal canal. Such rongeurs typically have a moveable cutting member that strikes a stationary plate.

Typically, a surgeon places bone and other biomaterial in an open region or mouth between a moveable member and a stationary strike plate; then the moveable member is translated against the tissue or other biological matter that in turn is compressed against the stationary plate. At this point the surgeon squeezes the handle of the rongeur so that the moveable member doses the mouth by further compressing the bio-matter against the stationary plate. This of course separates that bio-matter from the nearby tissue and bone. In most examples of a rongeur, the stationary plate is flat and the jaw is provided with peripheral cutting edges that bear directly against the flat plate at completion of the cutting motion,

Numerous alternative approaches to rongeur design have been proposed over the years. For example, U.S. Pat. No. 6,214,010 to Farley and herein incorporated by reference, teaches a rongeur that has a frame and a reciprocating member slidably associated with the frame. A blade is disposed on the distal end of the reciprocating member. The frame has a handle that is coupled with the reciprocating member for slideable movement of the reciprocating member. In this fashion, the blade is moved into a cutting position against a footplate formed in the distal end of the frame. A flexure is associated with the handle which allows the handle to be flexed. To reduce the potential for breakage of the footplate, a stop mechanism is provided for preventing further movement of the reciprocating member when the flexure is flexed. The patent to Farley teaches a handle attached to a main body via a pin 63 associated with a slot for activation of the handle mechanism. Prior Art FIG. 17 reproduces a similar arrangement as shown.

This drawing has a rear handle 2 disposed at an angle to and integrally formed with a shaft portion that extends out forwards and to the right in the figure. The juncture between the shaft and the handle 2 has a hole passing completely therethrough from the top to the bottom of the device; it should be easily understood that the two curved portions one on either side of the hole bound the right and left side of this hole. The hole permits the entry of the top of the pivoting forward handle 8 within this hole out and above the horizontal level of the shaft; this because the pivoting forward handle has a cutout at its end that is designed to engage a pin 15a integral with or inserted within the slide member sitting atop a guide rail integrally formed from the shaft.

As stated above, the pin 15a is either integral with or inserted within two corresponding holes on either side of the slide member as shown. If integral, it is formed from the inner surface of the slide member that has a space permitting this as a transverse device; otherwise, it is a separate component that is inserted in holes 15 on either side of the slide member for use as a male female screw combination for secure attachment thereto. Thus, the top cutout portion of the forward handle 8 acts against this pin 15a to move the slide member forwards as a hammer thereby cutting tissue, bone or otherwise. To complete the pivoting motion, the forward handle 8 is also attached to the two curved portions at the juncture between the shaft and the rear handle 2 using a pin male female screw attachment 13 (or the like) through corresponding holes 12a on both of the curved portions and a hole 12 on the handle 8. Finally, a user compresses the forward pivoting handle 8 on pin screw attachment 15a pivoting it on the screw attachment 13 thereby forcing the slide member at the top of the device down a rail in the shaft.

### Problems with this Approach

The type of cutting mechanism described heretofore, however, has inherent deficiencies. For example, the compression-type cut often results in splintering or fragmentation of the bone and or tissues that may be lost or dispersed into the surgical site. Additionally, the removed bone or tissue may become compacted into the rongeur mouth, potentially jamming the mechanism of the mouth preventing further actuation of the device. This is a severe maintenance problem and could lead to surgical complications as any cleaning of devices is time consuming and cumbersome. Further, the blades of the rongeur can become dulled after repeated uses or even pose a health threat if not properly disinfected.

Accordingly, there needs to be some solution to overcome the aforementioned problems.

### SUMMARY OF THE INVENTION

The present invention overcomes the deficiencies of the known art and the problems that remain unsolved by providing the following novelties.

A surgical rongeur comprising:
a main shaft integrated with
   a rear handle having an attachment for
a forward handle that cooperates with
a slide drive member loaded onto the main shaft and having an attached surgical blade.

In another aspect, wherein the surgical blade is a disposable blade.

In another aspect, wherein the surgical blade is substantially hollow.

In another aspect, wherein the surgical blade is attached to the slide drive member by a reusable mechanism.

In another aspect, wherein the surgical blade is attached to the slide drive member by a reversible mechanism.

In another aspect, wherein the surgical blade is removably attached to the slide drive member.

In another aspect, wherein said surgical blade further comprises:
a leading edge disposed at an angle such that the top portion of the blade is farther forward than the bottom portions of the blade.

In another aspect, wherein said surgical blade further comprises:
an end plate integral with the shaft having an obtuse angle to the shaft that corresponds with the leading edge of the blade thereby leaving no empty space there between.

In another aspect, wherein the surgical blade further comprises:
a perforation.

In another aspect, wherein the slide drive member further comprises:
a protrusion.

In another aspect, further comprising:
a perforation in the surgical blade that attaches it to
a protrusion on the slide drive member.

In another aspect, further comprising:
a recessed region of the slide drive member.

In another aspect, further comprising:
a rail integral with the top portion of the shaft formed for the moveable engagement of the slide drive member atop thereof.

In another aspect, wherein the shaft further comprises:
an integral ledge on both sides of the the rail.

In another aspect, wherein the slide drive member has a longitudinal depression along a portion of the underside thereof and formed for the moveable engagement atop the shaft thereto.

A surgical cutting tool comprising:
a main body having
   a rail and wherein the main body is movably attached to
a forward handle along a pivot point wherein the forward handle has another moveable attachment to
a striking device having
   a cutting tool removably connected thereto wherein the striking device is also movably attached to the main body along a slide.

In another aspect, wherein the the striking device has a corresponding depression that matches the contoured shaped of the rail thereby permitting the aforementioned to controllably slide upon the rail.

In another aspect, wherein the cutting tool is connected to a recessed portion of the striking device.

In another aspect, wherein the cutting tool has a perforation therein that matches a protrusion in the striking device.

A cutting blade comprising:
a parabolic longitudinal device having a
   perforation on a side thereof and
a front sharpened edge disposed at an angle such that the top of the parabolic curve is further forward than bottom curved legs thereof.
These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, in which:

FIG. 1 presents a right side elevation view of an assembled Surgical Rongeur with Disposable and Replaceable Cutting Blade in an embodiment herein.

FIG. 2 illustrates a right side elevation view of a disassembled Surgical Rongeur with Disposable and Replaceable Cutting Blade in an embodiment herein.

FIG. 3A describes a closeup right side elevation view of the front portion of the

Surgical Rongeur with Disposable and Replaceable Cutting Blade in an embodiment herein.

FIG. 3B presents a closeup right side isometric view of an assembled front portion of the Surgical Rongeur with Disposable and Replaceable Cutting Blade in an embodiment herein.

FIG. 3C illustrates a closeup right side isometric view of an a cutting blade used with the Surgical Rongeur in an embodiment herein.

FIG. 3D describes a closeup right side isometric view of the front portion of the slide used in the Surgical Rongeur in an embodiment herein.

FIG. 3E presents a closeup right side isometric view of the front portion of the base runner used in the Surgical Rongeur to mount the slide thereon in an embodiment herein.

FIG. 4 illustrates the cross section of cutting blade 20a as depicted by the dashed line 24 in FIG. 3C

FIG. 5 describes the cross section at the distal end of the slide drive member 7 where it receives the cutting blade 20a and is depicted by dashed line 25 in FIG. 3D.

FIG. 6 presents the cross section of the near distal end of the shaft 4 and is depicted by dashed line 26 in FIG. 3E.

FIG. 7 illustrates the cross section of the far distal end of the shaft 4 proximal to the end plate 5a along cross section 27.

FIG. 8 describes a side view of the mechanism of the surgical rongeur in an embodiment.

FIG. 9 presents a front cross section view along line 29 of FIG. 1 of the slide drive member in physical union with the shaft.

FIG. 10 illustrates a front cross section view of the far distal end of the shaft 4 proximal to the end plate 5a along cross section 27 of FIG. 3E; this view permits a showing of the flanges 5f but the rail 23b is no longer part of the profile.

FIG. 11 describes a front cross section view along line 30 of FIG. 3B showing of the blade placed atop the slide drive member in physical union with the shaft.

FIG. 12 presents a front cross section view along line 31 of FIG. 1 showing the blade front portion thereof atop the shaft.

FIG. 13 illustrates a front cross section view along line 32 of FIG. 2 showing the shaft.

FIG. 14 describes a front cross section view along line 28 of FIG. 2 showing the shaft having the central rail.

FIG. 15 presents a front cross section view along line 34 of FIG. 2 showing the forward portion of the slide drive member.

FIG. 16 illustrates a front cross section view along line 33 of FIG. 2 showing the blade.

FIG. 17 illustrates a handle portion of a prior art rongeur illustrating various connection points thereof.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in each figure. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

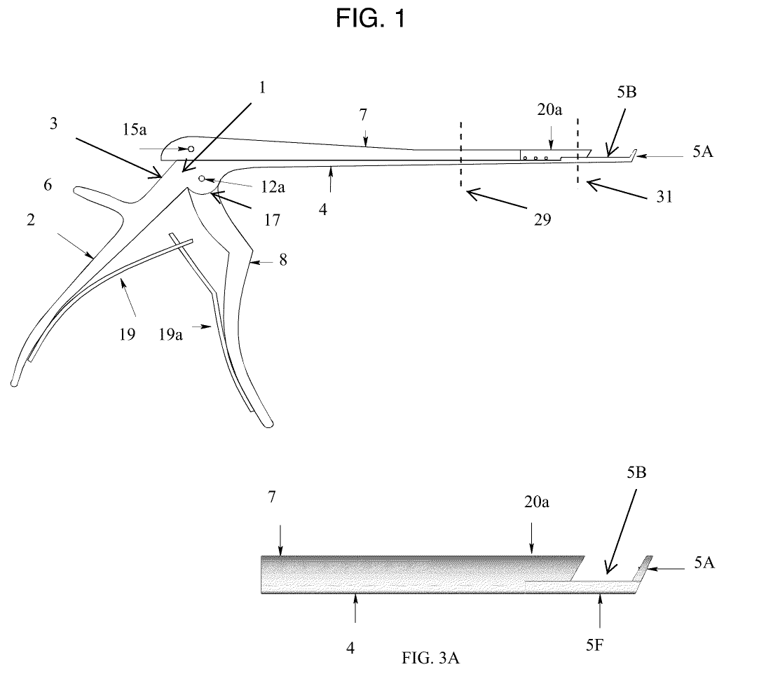

FIG. 1 presents a right side elevation view of an assembled Surgical Rongeur with Disposable and Replaceable Cutting Blade in an embodiment herein. This drawing has a rear handle 2 disposed at an angle to and integrally formed with a shaft 4 portion that extends out forwards and to the right in the figure. The juncture 17 between the shaft and the handle 2 has a hole passing completely therethrough from the top to the bottom of the device; it should be easily understood that the two curved portions one on either side of the hole bound the right and left side of this hole. The hole permits the entry of the top of the pivoting forward handle 8 within this hole out and above the horizontal level of the shaft; this because the pivoting forward handle has a cutout 11 at its end that is designed to engage a pin 15a integral with or inserted within the slide drive member 7 that is sitting atop a guide rail 18 integrally formed from the shaft.

As stated above, the pin 15a is either integral with or inserted within two corresponding holes on either side of the slide member as shown. If integral, it is formed from the inner surfaces of the slide member that has a space permitting this as a transverse device from the inner right side of the space to the inner left side therein; otherwise, it is a separate component that is inserted in holes 15 on either side of the slide member for use as a male female screw combination for secure attachment thereto.

Thus, the top cutout portion of the forward handle 8 acts against this pin 15a to move the slide drive member 7 forwards as a hammer thereby cutting tissue, bone or otherwise. To complete the pivoting motion, the forward handle 8 is also attached to the two curved portions at the juncture between the shaft and the rear handle 2 using a pin male female screw attachment 13 through holes 12a on both of the curved portions and corresponding hole 12 on the handle 8. Finally, a user compresses the forward pivoting handle 8 on pin 13 and against the pin 15a thereby forcing the slide member at the top of the device down a rail 11 in the shaft 7.

The improved surgical device shown herein comprises various items as described in the following. First, a curved body 1 having a hole passing from the top to bottom is integral with a rear handle 2 depending at an angle from the proximal end 3 of the body 1, and has a shaft 4 extending outwards and terminating at its distal end in an angled plate 5A. A support protrusion 6 extends from the upper portion of the rear handle 2; this helps a user maintain a proper grasp of the device as it provides support for the area of the hand between the thumb and the first finger. Mounted on the shaft 4 is a slide drive member 7 for reciprocating movement on the shaft 4.

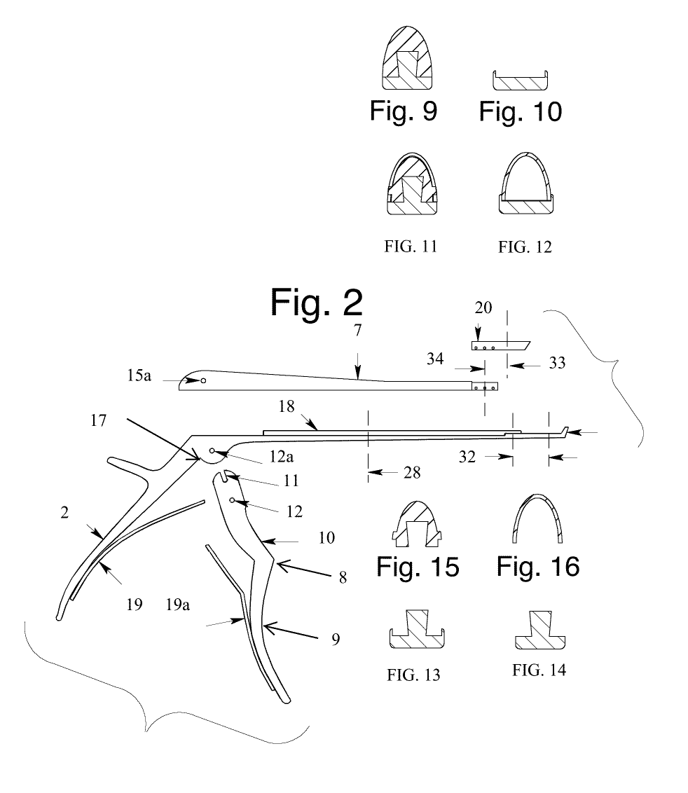

FIG. 2 illustrates a right side elevation view of a disassembled Surgical Rongeur with Disposable and Replaceable Cutting Blade in an embodiment herein. The figure shows a pivoting forward handle 8 (that is a separate part to be attached to main body 1) that includes a lower finger grip portion 9 and an upper finger portion 10 for a user's finger control of the device. The uppermost part of the forward handle 8 is a portion having a cutout 11 and a hole 12 (pin 13 inserted therein or integrally formed within) below it through which passes a pivot pin 13 such as a male female screw arrangement therethrough; the pivot pin 13 likewise passes through two corresponding holes 12a on the two curved under portions 17 of body 1.

It should be understood that holes 12a are located opposite each other such that the hole 12 on the forward handle 8 is located between the two corresponding holes 12a and the two curved under portions 17 are disposed laterally thereto for insertion of a pivot pin 13 screw arrangement; the pin 13 is typically made of two components male and female; one a threaded hollow cylinder for insertion of the male screw, but it should be understood that other types of attachments are possible. The portion of the handle 8 having the slot 11 fits into the body 1 through an hole extending top to bottom (not shown in the figure) in the region of the curved portion 17. This slot 11 grasps and engages a transverse integral member 15 formed within a space 16 (not shown) in the slide drive member 7. This transverse member 15 is formed integrally from the walls of the opening 16 of the slide member 7; thus, the transverse member extends from one inner side 16a to the other inner side 16b of the opening 16.

Alternatively, hole(s) 15a, running from one side to the other or on two sides having a hollow space therebetween in the slide member 7 permits the entry of a pivot pin 15 and or screw type arrangement (a male screw head with threaded body inserted in a female pin having an internally threaded cylinder) therein; this operates in conjunction with the slot 11 of the forward handle 8 that engages the pivot pin 15 (or transverse integral member) thereby permitting the motion of the forward handle that as a consequence actuates the slide drive member 7 to ride the rail 18 downline thereby cutting tissues forward of the device. In order to complete this action, one has to first attach the forward handle to the curved portion or main body 1 between the shaft 4 and the rear handle 2. The cutout slot 11 engages pin/member 15a located within the of slide drive member 7 that is mounted on the shaft 4 so that the forward handle 8 grabs the pin 15a and drives the slide drive member 7 forwards down the rail 18 as the forward handle 8 pivots on pin 13 within holes 12, 12a. This rail is integrally formed down the top center of the shaft 4 and begins forward of the juncture or main body 1 and ends down a portion of the operational plate 5b. Finally, the forward handle 8 and the rear handle 2, 3 are biased away from each other by spring means 19 and 19a.

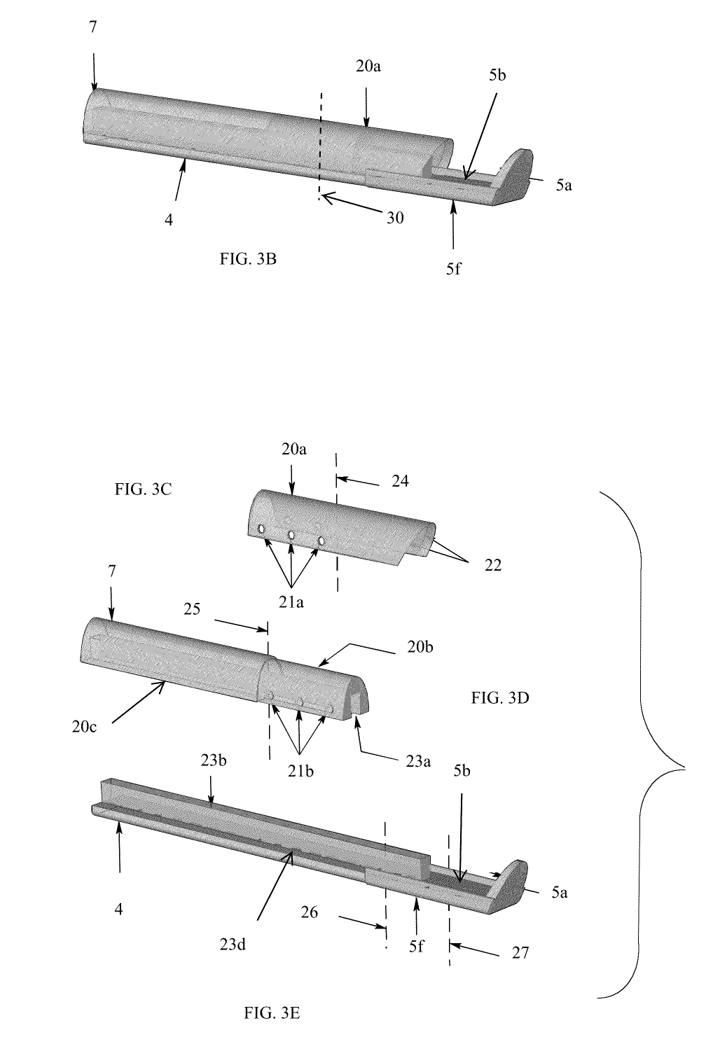

FIG. 3A describes a closeup right side elevation view of the front portion of the Surgical Rongeur with Disposable and Replaceable Cutting Blade in an embodiment herein. A slide drive member 7 sits atop the shaft 4 and is actuated by a user manipulating the handles to actuate a cutting movement whereby the slide and its associated blade 20a cuts through any bio-material placed in the space between the blade 20a and the end plate 5a on the operational plate 5b. It should be understood from the drawings that the end of the blade 20a and the end plate or anvil 5a forms a gapless interface there between. This end plate 5a is disposed at an obtuse angle (that is likewise matched by the forward part of the blade 20a ) to the operational plate 5b that juts out integrally and parallel thereto from the end of the shaft 4; moreover, two flanges 5f extend the operational plate 5b beyond the width of the slide drive member 7 as they reach outwards and upwards on either side thereof.

It should be understood that the cutting blade 20a is a disposable approximately upside down U shaped integral metal piece contoured to match the top surface of the end portion of the slide drive member 7. This end portion is otherwise known as the recessed end 20b of the slide drive member and has exposed protrusions that enable the easy attachment and removal of the blade 20a upon corresponding perforations in the blade portion 20a that engage the protrusions.

FIG. 3B presents a closeup right side isometric view of an assembled front portion of the Surgical Rongeur with Disposable and Replaceable Cutting Blade in an embodiment herein showing essentially the same components as one shown in FIG. 3A. FIGS. 3C, 3D, 3E together show the disassembled components comprising the front portions of the device having a shaft 4, a sliding member 7 and a cutting blade 20a.

FIG. 3C illustrates a closeup right side isometric view of an a cutting blade used with the Surgical Rongeur in an embodiment herein. The cutting blade 20a is a surgical metal piece shaped as an elongated thin parabolic curve formed from two arcs that unite in the center; further, the blade 20a is uniform longitudinally until it reaches the forward portion 22 thereof. At that point it is longer at the top of blade between the two curved parabolic legs than at the end of the two arcs; in other words, the arcs taper down so that the high point of the blade 20a is further forward than the endpoints of the two arcs. This identically matches the obtuse angular shape of the end plate 5a or anvil such that they form a gapless interface when juxtaposed. Finally, the two posterior right and left lateral surfaces of the blade 20a are perforated with three circular holes 21a linearly arranged near the bottom portion of the blade and on either side thereof for a total of six holes; these are to be used to attach the blade 20a to the forward recessed zone of the sliding member 7.

FIG. 3D describes a closeup right side isometric view of the front portion of the slide used in the Surgical Rongeur in an embodiment herein. It should be understood from the previous drawings that the slide drive member 7 is an elongated piece of surgical metal that starts as a gentle curved rounded shape and tapers until the approximate midpoint thereof at which point the device becomes linear in shape. This linear portion 20c proceeds forward until a breakpoint is reached whereupon the final portion of the slide drive member is formed as a recessed region 20b whose surface is smaller than the previous linear portion.

This recessed region 20b serves as a holder for the blade 20a that is attached by two sets of three laterally disposed protrusions 21b arranged linearly near the bottom edge of the recessed portion 20b. To complete the attachment of the two together, a user places the blade 20a near the recessed region 20b and opens the blade 20a slightly by hand and snaps it on the recessed region 20b such that the six holes 21a engage the protrusions 21b thereby locking the blade onto slide drive member 7. The blade 20a extends over and beyond the end of the recessed region 20b so as to be able to cut material between its forward leading edge 22 and the end plate 5a.

Finally, a sizable portion of the slide drive member 7 has a depression or trapezoidal type cutout (three sides of the trapezoid are visible the fourth side is virtual) that serves as a slide depression 23a to carry the aforementioned member 7 atop the rail 23b (18 in FIG. 1); to accomplish this, the rail is also a trapezoidal shape (three sides likewise visible whilst the fourth is integral with the rail) or any convenient shape that matches the depression 23a. The forward end of the depression 23a, the forward end of the recessed region 20b and the forward end of the rail 23b (18) can be placed together so that the forward portion of the recessed region 20b and the forward end of the rail 23b (18) form a fairly flat parallel surface; then it should be understood that this depression 23a begins at the end of the slide drive member 7 where the recessed region 20b is and proceeds backwards therein until somewhat after the matching rail 23b ends before the two curved under portions 17. Of course, the undersurface of the slide drive member is appropriately provided with any other necessary depressions to permit the forward motion of the slide drive member and the resetting thereof to its non-operational state whereby the slide drive member 7 is fully extended away from the end plate 5a. Finally, it is clear that a trapezoid shape was chosen here as it is larger at the top and tapers to the bottom on both sides so that the slide driver member 7 slide depression 23a does not disengage from the rail 23b.

FIG. 3E presents a closeup right side isometric view of the front portion of the base runner used in the Surgical Rongeur to mount the slide thereon in an embodiment herein. The bottom portion of the shaft 4 has a central longitudinal trapezoidal protrusion 23b or rail 18 disposed down the center of the shaft 4 thereby leaving space forming a ledge 23d on either side thereof; thus, along either side of the shaft or rail 23b there is a ledge 23d for the placement of the slide drive member 7 support surface 23c to rest thereon. The slide drive member 7 is loaded onto the rail 23b by inserting the posterior portion of the rail 23b into the forward opening of depression 23a where the recessed region 20b is located. It should be understood that this rail 23b and the shaft 4 is integrally formed along with the end plate 5a, operational plate 5b and flanges 5f previously described; further, it is clear that the rail 23b ends before the end plate 5a. This end plate 5a is disposed at an obtuse angle to the operational plate 5b that juts out integrally and parallel thereto from the end of the shaft 4; moreover, two flanges 5f extend the operational plate 5b beyond the width of the slide drive member 7 as they reach outwards and upwards on either side thereof.

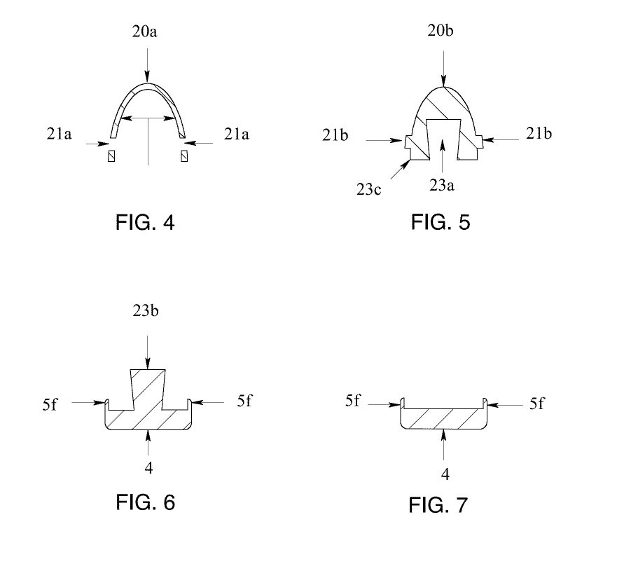

FIG. 4 illustrates the cross section of cutting blade 20a as depicted by the dashed line 24 in FIG. 3C. The inner face of the cutting blade 20a mates with the recessed face 20b when the cutting blade 20a is securely affixed to the slide drive member 7. The holes 21a are located on both sides of the cutting blade 20a. The holes 21a mate and nest with protrusions 21b on the slide drive member 7 when the cutting blade 20a is securely affixed to mobile shaft 7.

FIG. 5 describes the cross section at the distal end of the slide drive member 7 where it receives the cutting blade 20a and is depicted by dashed line 25 in FIG. 3D. The recessed face 20b is the outer profile of the cross section. The protrusions 21b linearly disposed are shown on both sides of the slide drive member 7. The slide depression 23a is seen cut through the bottom surface of the slide drive member 7. It should be noted that the slide drive member 7 has a slide depression 23a that is symmetrically trapezoidal. FIG. 5 also illustrates the slide drive members support surface 23c that rest upon the ledge 23d on either side of the rail 23b.

FIG. 6 presents the cross section of the near distal end of the shaft 4 and is depicted by dashed line 26 in FIG. 3E. The rail 23b is seen to have an outer profile that mates with depression 23a. The flanges 5f are illustrated on either side of the forward portion of the shaft 4 and form a channel for the support surface 23c of the slide drive member 7.

FIG. 7 illustrates the cross section of the far distal end of the shaft 4 proximal to the end plate 5a along cross section 27; this view permits a showing of the flanges 5f but the rail 23b is no longer part of the profile.

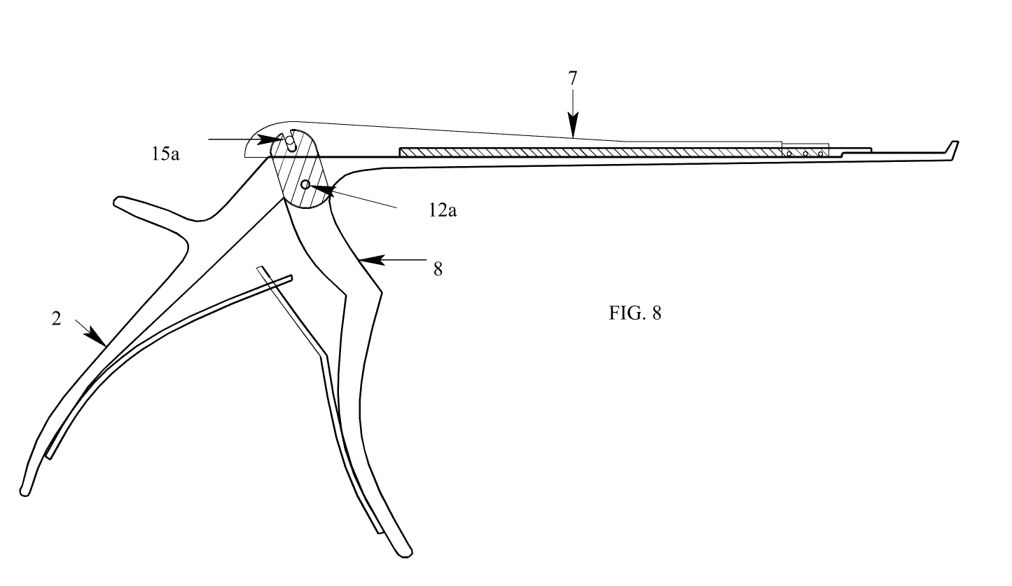

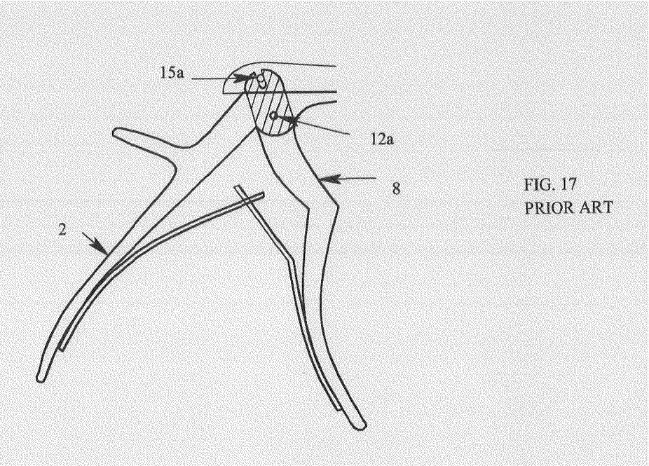

FIG. 8 describes a side view of the mechanism of the surgical rongeur in an embodiment.

FIG. 9 presents a front cross section view along line 29 of FIG. 1 of the slide drive member in physical union with the shaft.

FIG. 10 illustrates a front cross section view of the far distal end of the shaft 4 proximal to the end plate 5a along cross section 27 of FIG. 3E; this view permits a showing of the flanges 5f but the rail 23b is no longer part of the profile.

FIG. 11 describes a front cross section view along line 30 of FIG. 3B showing of the blade placed atop the slide drive member in physical union with the shaft.

FIG. 12 presents a front cross section view along line 31 of FIG. 1 showing the blade front portion thereof atop the shaft.

FIG. 13 illustrates a front cross section view along line 32 of FIG. 2 showing the shaft.

FIG. 14 describes a front cross section view along line 28 of FIG. 2 showing the shaft having the central rail.

FIG. 15 presents a front cross section view along line 34 of FIG. 2 showing the forward portion of the slide drive member.

FIG. 16 illustrates a front cross section view along line 33 of FIG. 2 showing the blade.

### CONCLUSION

Dull surgical instruments put the patient at risk due to ineffective and imprecise removal of tissue. Further, blunt surgical tools tear tissue and can damage structures and tissues not intended to be damaged. Thus, these are ineffective at removing tissue and as a consequence they can increase surgical time and the effort a surgeon makes in completing an operation. It is very common indeed that during the course of a surgical procedure cutting surgical tools become dulled and need to be changed during the course of a single operation. It is for this reason that an easy, effective and reproducible way is needed to maintain the sharpness of surgical rongeurs used in spinal surgery and other similar procedures that require an effective cutting device. This problem has been solved in the instant description and has been enabled in the Surgical Rongeur with Disposable and Replaceable Cutting Blade description and claimed below.

Further, the improved cutting mechanics and reduction of forces at the anvil allow for a smaller anvil that decreases the intrusion of the surgical tool into delicate neural tissues. This promotes the current trend in new techniques being used in spinal surgery that are aimed at reducing surgical trauma by reducing surgical exposures; a small exposure needs less bulky tools.

The above-described embodiments are merely exemplary illustrations of implementations set forth for a clear understanding of the principles of the invention. Many variations, combinations, modifications or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

I claim:

1. A surgical rongeur comprising:
a main shaft integrated with
a rear handle having an attachment for
a forward handle that cooperates with
a slide drive member loaded onto the main shaft and having an attached surgical blade.

2. The surgical rongeur of claim 1, wherein the surgical blade is a disposable blade.

3. The surgical rongeur of claim 1, wherein the surgical blade is substantially hollow.

4. The surgical rongeur of claim 1, wherein the surgical blade is attached to the slide drive member by a reusable mechanism.

5. The surgical rongeur of claim 1, wherein the surgical blade is attached to the slide drive member by a reversible mechanism.

6. The surgical rongeur of claim 1, wherein the surgical blade is removably attached to the slide drive member.

7. The surgical rongeur of claim 1, wherein said surgical blade further comprises:
a leading edge disposed at an angle such that the top portion of the blade is farther forward than the bottom portions of the blade.

8. The surgical rongeur of claim 7, wherein said surgical blade further comprises:
an end plate integral with the shaft having an obtuse angle to the shaft that corresponds with the leading edge of the blade thereby leaving no empty space there between.

9. The surgical rongeur of claim 1, wherein the surgical blade further comprises:
a perforation.

10. The surgical rongeur of claim 1, wherein the slide drive member further comprises:
a protrusion.

11. The surgical rongeur of claim 1, further comprising:
a perforation in the surgical blade that attaches it to
a protrusion on the slide drive member.

12. The surgical rongeur of claim 1, further comprising:
a recessed region of the slide drive member.

13. The surgical rongeur of claim 1, further comprising:
a rail integral with the top portion of the shaft formed for the moveable engagement of the slide drive member atop thereof.

14. The surgical rongeur of claim 13, wherein the shaft further comprises:
an integral ledge on both sides of the the rail.

15. The surgical rongeur of claim 1, wherein the slide drive member has a longitudinal depression along a portion of the underside thereof and formed for the moveable engagement atop the shaft thereto.

16. A surgical cutting tool comprising:
a main body having
a rail and wherein the main body is movably attached to
a forward handle along a pivot point wherein the forward handle has another moveable attachment to
a striking device having
a cutting tool removably connected thereto wherein the striking device is also movably attached to the main body along a slide.

17. The surgical cutting tool of claim 16, wherein the the striking device has a corresponding depression that matches the contoured shaped of the rail thereby permitting the aforementioned to controllably slide upon the rail.

18. The surgical cutting tool of claim 16, wherein the cutting tool is connected to a recessed portion of the striking device.

19. The surgical cutting tool of claim 18, wherein the cutting tool has a perforation therein that matches a protrusion in the striking device.

20. A cutting blade comprising:
a parabolic longitudinal device having a
perforation on a side thereof and
a front sharpened edge disposed at an angle such that the top of the parabolic curve is further forward than bottom curved legs thereof.
